(19) United States
(12) Patent Application Publication
(10) Pub. No.: US 2006/0009744 A1
(43) Pub. Date: Jan. 12, 2006

(54) Decorative component for an absorbent article

(76) Inventors: Edward P. Erdman, West Chester, PA (US); Carol L. Erdman, West Chester, PA (US)

Correspondence Address: Christopher C. Campbell, Esq.;Hunton & Williams, Suite 1200, 1900 K Street, NW, Washington, DC, 20006-1109, US

(21) Appl. No.: 10/886,712

(22) Filed: Jul. 9, 2004

(51) Int. Cl. A61F 13/15

(52) U.S. Cl. 604/385.01

(57) ABSTRACT

Decorative components of various sizes and uses for assembly into absorbent articles are manufactured using a decorative component material. The absorbent articles have a variety of decorative appearances. A method for producing absorbent articles from the decorative component material, as well as a method for preparing a package of absorbent articles having varied outer covers, is also disclosed.

### FIELD OF THE INVENTION

The invention generally relates to decorative components for absorbent articles, such as disposable diapers, adult incontinence products, training pants and the like. In particular, the invention relates to decorative components prepared from decorative materials for use in the continuous manufacture of absorbent articles, a method of manufacturing absorbent articles incorporating the decorative component such that the articles exhibit a variety of decorative appearances, and a method of preparing a package of absorbent articles having a variety of decorative appearances. In addition, the same decorative material may be used to prepare a variety of decorative components that provide absorbent articles having a variety of decorative appearances.

### BACKGROUND OF THE INVENTION

Absorbent articles, such as diapers, adult incontinence products, training pants, and feminine care products, are in widespread use today. Such absorbent articles typically include a moisture pervious top sheet that is located substantially next to the body of the wearer, an absorbent core, and a moisture impervious back sheet that is located on or near the outwardly facing part (the “outer cover”) of the article.

Absorbent articles typically are prepared by continuously supplying many of the various components of the article, and assembling these components into the final article. For example, the top sheet material and the back sheet material may be continuously supplied as web material in a machine direction of an assembly line. Absorbent cores, along with other features of the article as elastic elements, may be disposed at predetermined positions on components of the absorbent article during assembly. The various components may then be associated to form a composite, and the composite may be cut at predetermined locations to form the final absorbent article.

Manufacturers of absorbent articles are known to incorporate decorations such as colors and designs into certain components of the articles to provide a pleasing decorative appearance for consumers, both in the package and during use. In addition, manufacturers may package absorbent articles that have a variety of decorative appearances in a single package for the consumer's enjoyment and variety.

To prepare decorative absorbent articles, a common procedure is to print graphics on a decorative component material and prepare a decorative component of the absorbent article from that material. For example, a back sheet material, a fastening mechanism material, or any other component material for preparing a component that is visible from the outside of the absorbent article may be printed with one or more designs, which may continuously transition over the decorative component material. The design contained on a decorative component material may be comprehensive or noncomprehensive. Alternatively, numerous different comprehensive designs may be contained on the roll. The term “comprehensive” as it relates to a design refers to a design that appears to be a single, complete unit with a clear cut beginning and end, such as a single character or group of characters or some other type of graphic which appears to be intended to be displayed as a single, complete unit. A “noncomprehensive” design refers to a design that does not appear to be a single, complete unit, but rather does not appear to have a clear cut beginning or end and does not appear to be intended to be displayed as a single unit.

As noted, the decorative component material is typically continuously supplied during assembly of the absorbent article. In the case of a noncomprehensive design, where there is no discernible beginning or end of the design, there may be no need to coordinate the design with the positioning of the decorative component on the absorbent article, because the design appears essentially the same regardless of where it is positioned on the component. Such a design has been referred to as an “unregistered” graphic, as no steps are taken during the assembly process to position the decorative component on the absorbent article such that the component displays the design in a certain position.

Systems and apparatuses have been developed that are capable of coordinating a comprehensive design with the positioning of the decorative component on a web material, such as is used to assemble an absorbent article. U.S. Pat. Nos. 5,235,515 and 5,286,543, both issued to Ungpiyakul et. al., the disclosures of which are hereby incorporated in their entireties in a manner consistent with this application, describe the use of registration marks placed on a decorative component material, where the registration marks are positioned in a known, predetermined relationship with the design. Sensors are incorporated into the assembly line that are capable of sensing the registration marks, and controllers can time the cutting and placement of the decorative component material to the continuously supplied web material such that a single comprehensive design is displayed on the decorative component as assembled in the absorbent article. Such uses of registration marks to provide a continuously supplied material in a certain relationship to another continuously supplied material is commonly known as “registered” graphics.

Thus, absorbent articles having desirable decorative appearances may be prepared by the use of decorative component materials containing either comprehensive or noncomprehensive designs. The manufacture of absorbent articles having different decorative appearances using rolls of decorative component materials containing noncomprehensive designs requires either multiple assembly lines, each assembly line using rolls of decorative component materials with different noncomprehensive designs, or different runs on the same assembly line, each run using rolls of decorative component materials with different noncomprehensive designs.

Packages of absorbent articles with a variety of desirable decorative appearances may be prepared from rolls of decorative component materials having comprehensive designs through the use of registration marks on the materials. Where a single comprehensive design is contained on the material, the production of a variety package requires either multiple assembly lines or multiple runs on one assembly line, as with noncomprehensive designs, and manual assembly or the use of specialized equipment to combine products from the multiple runs to form the variety package. Variety packages of absorbent articles having desirable decorative appearances may be manufactured using rolls of decorative component materials having multiple comprehensive designs and registration marks, if the assembly process includes the attendant instruments and equipment to time the cutting and placement of the decorative component material to the web material using the registration marks.

### SUMMARY OF THE INVENTION

It would be desirable to provide a decorative component material from which absorbent articles with a variety of desirable decorative appearances can be continuously manufactured. It would further be desirable to provide a single roll of a decorative component material that can be used to manufacture decorative components of various sizes and shapes, where the absorbent articles assembled using those components have a variety of desirable decorative appearances.

The preferred embodiments are intended to provide these desirable features, plus additional benefits. It is therefore a feature of the preferred embodiments to provide a roll of decorative component material from which absorbent articles having a variety of decorative appearances can be manufactured. It is further a feature of the preferred embodiments to provide a single roll of decorative component material from which decorative components of different sizes can be prepared for assembly into absorbent articles having a variety of desirable decorative appearances.

In accordance with these and other features of various embodiments of the invention, there is provided a roll of decorative component material comprising a design from which absorbent articles having a variety of desirable decorative appearances can be manufactured on a single assembly line. In one exemplary embodiment, the roll of decorative component material comprises one or more noncomprehensive designs that, upon assembly of a component made from the material into an absorbent article, provide a different decorative appearance to each successive article made in continuous assembly. The design may, but does not have to, repeat along the longitudinal dimension of the decorative component material (the “pattern length”), but any pattern length could preferably be selected such that it does not coincide with either one or both of the longitudinal and lateral length of a component made from the roll of decorative component material. Thus, absorbent articles with different desirable decorative appearances can be manufactured on a single assembly line using components made from the roll of decorative component material. In this embodiment, the pattern length would preferably, but not necessarily, either be greater than or less than, but not the same as, either one or both of the longitudinal and lateral length of the component made from the roll of decorative component material.

In another exemplary embodiment, the roll of decorative component material comprises one or more noncomprehensive designs that, once the decorative component is assembled into an absorbent article, provide a decorative appearance to the article. The designs may, but do not have to, repeat along the longitudinal dimension of the decorative material, but the pattern length should preferably, but not necessarily, be selected such that it does not coincide with either one or both of the longitudinal and lateral lengths of several different decorative components that may be produced from the roll of decorative component material for assembly into absorbent articles of different sizes and/or designs. Thus, absorbent articles having a variety of decorative appearances, but of different sizes and/or designs, can be manufactured using the same roll of decorative component material. In this embodiment, the versatility of the roll of decorative component material is enhanced.

The noncomprehensive design(s), according to the preferred embodiments, may be placed on the decorative component material using any process now known or later developed to incorporate decorations into such materials, including but not limited to the offset printing process and programmable computerized printing.

In yet another exemplary embodiment, a method for preparing a package of absorbent articles substantially directly from an assembly line is disclosed, where the package contains absorbent articles having a variety of desirable decorative appearances.

These and other features and benefits of the invention will become apparent to those skilled in the art from the following detailed descriptions.

### BRIEF DESCRIPTION OF THE DRAWINGS

FIG. 1 is a partially cut-away view of an embodiment of the invention, shown in the fully-flattened position;

FIGS. 2a and 2b are views of a back sheet made from a decorative component material with a design according to one embodiment of the invention, with the profile of cut-outs for finished absorbent articles overlaying the views;

FIG. 3 is a view of one embodiment of the decorative component material according to the invention; and

FIG. 4 is a view of another embodiment of the decorative component material according to the invention.

### DETAILED DESCRIPTION OF THE PREFERRED EMBODIMENTS

The terms “absorbent article” or simply “article” refer to devices that absorb and contain body fluids and other body exudates. More specifically, these terms refer to articles that are placed against or in proximity to the body of a wearer to absorb and contain the various exudates discharged from the body. A non-exhaustive list of examples of absorbent articles includes diapers, diaper covers, disposable diapers, training pants, feminine hygiene products and adult incontinence products. Such articles may be intended to be discarded or partially discarded after a single use (“disposable” articles). Such articles may comprise essentially a single inseparable structure (“unitary” articles), or they may comprise replaceable inserts or other interchangeable parts.

The preferred embodiments may be used with all of the foregoing classes of absorbent articles, without limitation, whether disposable or otherwise. The embodiments described herein provide, as an exemplary structure, a diaper for an infant, however this is not intended to limit the claimed invention. The preferred embodiments will be understood to encompass, without limitation, all classes and types of absorbent articles, including those described herein.

The expressions “upper layer,” “lower layer,” “above” and “below,” which refer to the various components included in the absorbent core and other components of the absorbent article (including the layers surrounding the absorbent core, if present) are used merely to describe the spatial relationship between the respective components. The upper layer or component “above” the other component need not always remain vertically above the core or component, and the lower layer or component “below” the other component need not always remain vertically below the core or component.

The term “component” can refer, but is not limited, to designated selected regions, such as edges, corners, sides or the like; structural members, such as elastic strips, absorbent pads, stretchable layers or panels, layers of material, or the like; or a graphic.

The term “disposed” and the expressions “disposed on,” “disposing on,” “disposed in,” “disposed between” and variations thereof (e.g., a description of the article being “disposed” is interposed between the words “disposed” and “on”) are intended to mean that one element can be integral with another element, or that one element can be a separate structure bonded to or placed with or placed near another element. Thus, a component that is “disposed on” an element of the absorbent article can be formed or applied directly or indirectly to a surface of the element, formed or applied between layers of a multiple layer element, formed or applied to a substrate that is placed with or near the element, formed or applied within a layer of the element or another substrate, or other variations or combinations thereof.

The terms “top sheet” and “back sheet” denote the relationship of these materials or layers with respect to the absorbent core. It is understood that additional layers may be present between the absorbent core and the top sheet and back sheet, and that additional layers and other materials may be present on the side opposite the absorbent core from either the top sheet or the back sheet.

The term “decorative component material” means a material or materials, or a laminate of materials, used for a component of an absorbent article that lends a decorative appearance to the article. The material may be of different compositions depending on the component. For example, if the component is a back sheet, the component material may be a liquid impervious polyethylene, polypropylene, polyester, nylon, polyvinyl chloride, polylactic acid or blends of these materials. If the component were an outer cover for the back sheet, it may comprise a fibrous nonwoven fabric such as cellulosic fibers, or polyethylene, polypropylene, polyester, nylon, polyvinyl chloride, polylactic acid or blends of these materials. At least one noncomprehensive design will be incorporated into the decorative component.

The term “noncomprehensive design” denotes a graphic representation of any combination of colors and designs that does not appear to be a single, complete unit, but rather appears to have no clear cut beginning or end, and does not appear to be intended to be displayed as a single unit. Noncomprehensive designs may include patterns that repeat with no clear unit of repeat, or may not repeat, or may have a repeat large enough that they do not appear to repeat. Noncomprehensive designs include designs that may comprise multiple units of other designs.

The term “pattern length” denotes the length, measured along the longitudinal dimension of a decorative component material, of a single design, if present. The design repeats after each pattern length.

For clarity, features that appear in more than one Figure have the same reference number in each Figure.

The preferred embodiments relate to a decorative component material used in the continuous manufacture of an absorbent article. The decorative component material has a longitudinal dimension and a lateral dimension, and comprises at least one noncomprehensive design along at least its longitudinal dimension. The decorative component material is provided continuously in a machine direction substantially along its longitudinal dimension to an assembly line for the manufacture of decorative components for assembly into absorbent articles. The design is disposed on the decorative component material such that, upon assembly of a decorative component made from the material into an absorbent article, it tends to provide the article with a desirable decorative appearance. The design is disposed on the decorative component material in at least the longitudinal direction at a pattern length such that successive decorative components manufactured on a continuous assembly line using components comprising the decorative component material will display different desirable decorative appearances, for assembly into absorbent articles having different decorative appearances.

Absorbent articles with a variety of desirable decorative appearances may be manufactured in one embodiment by using a decorative component material that includes at least 2 or more colors, preferably 3 to 6 colors, more preferably 3 to 5 colors, and even more preferably 4 colors in each design. The colors included in the design are preferably complementary, and may blend into each other to provide smooth transitions to provide a pleasing appearance.

In another preferred embodiment, the pattern length of the design in at least the longitudinal dimension of the decorative component material should preferably, but not necessarily, be less than or greater than, but not equal to, either one or both of the longitudinal and lateral length of each decorative component made from the material. For example, the back sheet for a large sized diaper may have a longitudinal length of approximately 49 cm. The pattern length of a design contained in a decorative component material used to supply material for the back sheet should preferably, but not necessarily, be less than 49 cm, or greater than 49 cm. If the pattern length of a design was 75 cm, for example, each individual design would exceed the longitudinal length of the component made from the decorative component material. Alternatively, if the pattern length of the design was 20 cm, more than one design is incorporated into the back sheet of each absorbent article made from the decorative component material. Successive absorbent articles made using back sheets prepared from this same decorative component material, where the longitudinal dimension of the decorative component material substantially corresponds to the longitudinal dimension of the absorbent article, will then have different decorative appearances.

Alternatively, it may be desirable to provide a back sheet prepared from this decorative component such that the longitudinal dimension of the decorative component material corresponds substantially to the lateral dimension of the absorbent article, in which case the pattern length should preferably, but not necessarily, be greater than or less than, but not equal to, the lateral length of the back sheet according to the invention. Also contemplated are absorbent articles prepared using decorative components other than back sheets, such as, for example, fastening mechanisms. Using the guidelines provided herein, those of ordinary skill in the art can design a decorative component material with pattern lengths different from either one or both of the longitudinal and lateral lengths of a component made using that material without undue experimentation, regardless of whether the decorative component is disposed with either of its longitudinal or lateral dimension substantially corresponding to the longitudinal dimension of the absorbent article.

One of the advantages of a decorative component material with the described attributes is that the same roll of decorative component material may be used in the manufacture of a variety of types and sizes of components while still providing the advantages of the preferred embodiments. The pattern length of the design can be easily determined by one of ordinary skill in the art by determining the different longitudinal and/or lateral lengths of components intended to be produced using the particular decorative component material, and thus establishing the pattern length to be less than or greater than any of these longitudinal and/or lateral lengths, and also not a multiple of any of these longitudinal and/or lateral lengths.

Another preferred embodiment relates to a method for producing decorative components of various sizes for assembly into absorbent articles that provide a variety of decorative appearances to the articles from a single roll of decorative component material. The pattern length of the design on the decorative component material is calculated so that it preferably, but not necessarily, does not coincide with at least one of the longitudinal and lateral lengths of any of the components made from that material, and therefore each successive absorbent article that is assembled with components made from the same decorative component material will have a different decorative appearance than either the previous or the successive article. Because the colors in the designs incorporated into the decorative component material are preferably complementary and blend into each other, the resulting absorbent articles will have a variety of pleasing decorative appearances. The use of a decorative component material prepared in this manner thus allows absorbent articles with a variety of decorative appearances to be continuously manufactured on one assembly line.

According to yet another preferred embodiment, a method of preparing packages of absorbent articles having a variety of desirable decorative appearances can be accomplished by manufacturing the articles using a decorative component material prepared in the above-described manner and packaging the articles substantially directly after they leave the assembly line.

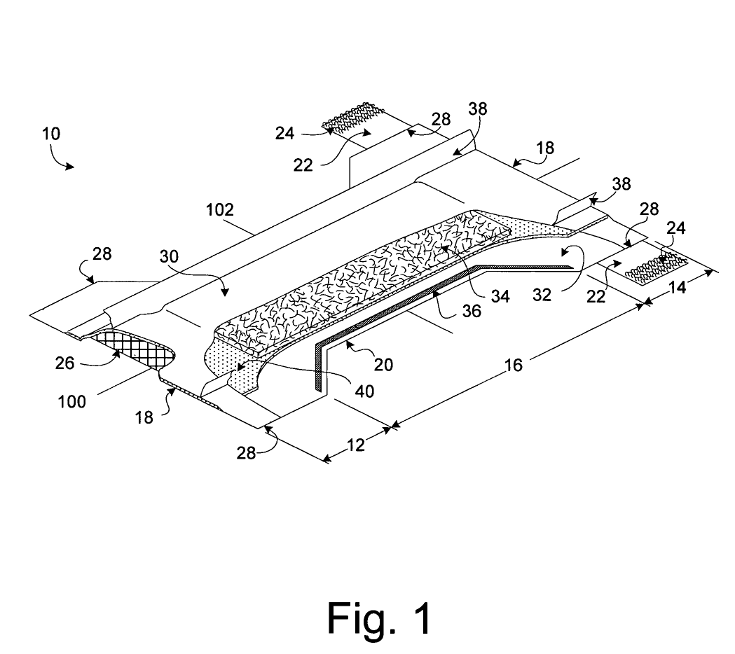

Turning now to the Figures, FIG. 1 is a partially cut away depiction of an exemplary absorbent article 10 according to the preferred embodiments. The embodiment shown in FIG. 1 is an infant's diaper, however, this depiction is not intended to limit the invention. The article 10 of FIG. 1 is depicted in a flattened position, with the various elastic components depicted in their extended position for clarity. In the flattened position, the article 10 generally has an hourglass shaped structure, but it may also have a rectangular, trapezoidal or other shape.

As used herein, the longitudinal axis 100 of the article is the dimension of the article corresponding to the front-to-rear dimension of the user, and the lateral axis 102 of the article is the dimension corresponding to the side-to-side dimension of the user.

In use, an embodiment of the invention may comprise a pant-like article 10 having a waist-encircling region and a crotch region. The waist-encircling region may comprise a front region 12, corresponding with the front of a wearer's body, and a rear region 14, corresponding with the back of a wearer's body, that are joined together at or near their lateral edges 28, causing the longitudinally distal edges 18 of the article 10 to form the perimeter of a waist opening. The crotch region 16 extends between the front and rear regions 12, 14, and the crotch edges 20 form the perimeter of a pair of leg openings.

The front and rear regions 12, 14 may be joined to one another by permanent seams (not shown) or by releasable fasteners 22. The releasable fasteners 22 may comprise an adhesive tape, a mechanically interlocking fastener, such as a hook and loop fastener 24 or a button or snap, or any other suitable releasable fastening device. The article also may comprise a target surface 26 associated with a surface of the article 10, that is selected to interact with the releasable fasteners 22 to provide them with the grip necessary to hold the article 10 together. While the embodiment depicted in FIG. 1 shows the releasable fasteners 22 being located in the rear region 14, and the target surface 26 being in the front region 12, one of ordinary skill in the art will recognize that these locations may be reversed. The selection and manufacture of permanent side seams, releasable fasteners 22, and target surfaces 26 is known in the art, and one of ordinary skill in the art will be able to implement such structures and devices without undue experimentation.

The article 10 preferably comprises a top sheet 30, and a back sheet 32, which may be substantially coterminous with the top sheet 30. When the article 10 is being worn, the top sheet 30 faces the wearer's body, and the back sheet 32 faces away from the wearer. An absorbent core 34 usually is disposed between at least a portion of the top sheet 30 and at least a portion of the back sheet 32. Components, such as a barrier layer 40, may be disposed between the absorbent core 34 and the back sheet 32. Other components, such as a transfer layer (not pictured), may be disposed between the absorbent core 34 and the top sheet 30. Preferably, the absorbent core 34 is disposed between at least a portion of the top sheet 30 and barrier layer 40.

A preferred embodiment of the invention may further comprise various additional features. One or more pairs of leg elastics 36 may extend adjacent the crotch edges 20. The article 10 may also comprise one or more waste containment systems, such as a pair of standing leg gathers 38. The standing leg gathers 38 preferably extend longitudinally between the front region 12 and the rear region 14 along opposite sides of the article's longitudinal axis 100.

The various parts of the article 10 are associated with one another to form a structure that preferably maintains its shape during the useful life of the article 10. As used herein, the term “associated” encompasses configurations whereby a first part is directly joined to a second part by affixing the first part directly to the second part, by indirectly joining the first part to the second part through intermediate members, or by fixing the relative positions of various parts by capturing parts between other parts. Those skilled in the art will appreciate that various methods or combinations of methods may be used to securely join the parts of the article 10.

These features, and other features, functions and uses of the invention, are described in greater detail herein.

The top sheet 30 and back sheet 32 may be constructed from a wide variety of materials known in the art. The invention is not intended to be limited to any specific materials for these components. The top sheet 30 and back sheet 32 may be shaped and sized according to the requirements of each of the various types of absorbent article, or to accommodate various user sizes. Where the preferred embodiment is a diaper or an adult incontinence brief, the top sheet 30, back sheet 32, or both, may have an hourglass shape, as seen in FIG. 1, or may have a rectangular, trapezoidal, “T” shape, or other shape.

The back sheet 32 generally is made of any suitable pliable liquid impervious material known in the art or later discovered. Typical back sheet materials include films of polyethylene, polypropylene, polyester, nylon, and polyvinyl chloride and blends of these materials. For example, the back sheet 32 may be made of a polyethylene film having a thickness in the range of 0.01-0.03 mm. The back sheet 32 may be pigmented with, for example, titanium dioxide, to provide the article 10 with a pleasing color or to render the back sheet 32 opaque enough that exudates being contained by the article 10 are not visible from outside the article. In addition, back sheet 32 may be formed in such a manner that it is opaque, for example, by using various inert components in the polymeric film. The back sheet 32 may be prepared from a decorative component material according to the invention. Other back sheet materials will be readily apparent to those of ordinary skill in the art. The back sheet 32 preferably should have sufficient liquid imperviousness to prevent any leakage of fluids through the back sheet 32. The required level of liquid imperviousness may also vary between different locations on the article 10.

The back sheet 32 may further comprise separate regions having different properties. In a preferred embodiment, portions of the back sheet 32 are air-permeable to improve the breathability, and therefore comfort, of the article 10. The different regions may be formed by making the back sheet 32 a composite of different sheet materials. Other regions may undergo chemical treatment, pressure treatment, heat treatment, adhesive application, or other processes or methods known in the art to provide desirable properties as known to those of ordinary skill in the art. Some regions of the back sheet 32 may be fluid pervious. In one embodiment of the invention, the back sheet 32 is fluid impervious in the crotch 12, but is fluid pervious in portions of the front and rear regions 12, 14. The back sheet 32 may also be made from a laminate of overlaid sheets of material.

The back sheet 32 may be covered with a fibrous, nonwoven fabric such as is disclosed, for example, in U.S. Pat. No. 4,646,362 issued to Heran et al., which is hereby incorporated by reference in its entirety and in a manner consistent with the present application and invention. Materials for such a fibrous outer liner include a spun-bonded nonwoven web of synthetic fibers; a nonwoven web of cellulosic fibers, textile fibers, or a blend of cellulosic and textile fibers; a spun-bonded nonwoven web of synthetic fibers mixed with cellulosic, pulp fibers, or textile fibers; and melt blown thermoplastic fibers or mixtures of such thermoplastic fibers with cellulosic, pulp or textile fibers. The outer liner may have a surface that comprises the outer cover of the absorbent article 10, and upon which a color and/or design scheme may be disposed according to the preferred embodiments.

The moisture-pervious top sheet 30 may be made of any suitable relatively liquid-pervious material currently known in the art or later discovered that permits passage of a liquid therethrough. Examples of suitable top sheet materials include nonwoven spun-bonded or carded webs of polypropylene, polyethylene, nylon, polyester and blends of these materials, perforated, apertured, or reticulated films, and the like. Nonwoven materials are exemplary because such materials readily allow the passage of liquids to the underlying absorbent core 34. The top sheet 30 preferably comprises a nonwoven material that may be made of carded fibers, either adhesively or thermally bonded, perforated or apertured film, spunbonded fibers, or water entangled fibers, which generally weigh from about 10 to about 30 grams/m² and have appropriate and effective machine direction and cross-machine (transverse) direction strength suitable for use as a top sheet material for the given application. The invention is not intended to be limited to any particular material for the top sheet 30, and other top sheet materials will be readily apparent to those of ordinary skill in the art.

The top sheet 30 may further comprise several regions having different properties. In one embodiment of the invention, the laterally distal portions of the top sheet 30 are preferably substantially fluid impervious and hydrophobic, while the remainder of the top sheet 30 is hydrophilic and fluid pervious. Different top sheet properties, such as fluid perviousness and hydrophobicity, may be imparted upon the top sheet 30 by treating the top sheet 30 with adhesives, surfactants, or other chemicals, using a composite of different materials, or by other means. The top sheet 30 also may be made from a laminate of overlaid sheets of material. The top sheet 30 also may be treated in specific areas like the crotch region, with skin wellness ingredients like aloe and vitamin E.

As noted elsewhere herein, the top sheet 30 and back sheet 32 may be substantially coterminous, or they may have different shapes and sizes. The particular design of the top sheet and back sheet may be dictated by manufacturing considerations, cost considerations, and performance considerations. Preferably, the top sheet 30 is large enough to completely cover the absorbent core 34, and either the back sheet 32 or the barrier layer 40 are large enough to prevent leakage from the article 10. The design of top sheets 30 and back sheets 32 is known in the art, and a skilled artisan will be able to produce an appropriate top sheet 30 and an appropriate back sheet 32 without undue experimentation.

The top sheet 30 and the back sheet 32 may be associated with one another using a variety of methods known in the art. For example, they may be thermally, ultrasonically, or chemically bonded to one another. They also may be joined using lines of hot melt adhesive or mechanical fasteners, such as thread, clips, or staples. In one embodiment, a hydrophilic adhesive, such as those sold by National Starch and Chemical Company of Bridgewater, N.J. under the trademark CYCLOFLEX, is used to join the top sheet 30 to the back sheet 32. The particular joining method may be dictated by the types of materials selected for the top sheet 30 and back sheet 32.

An absorbent core 34 preferably is disposed between the top sheet 30 and the barrier layer 40 or back sheet 32 in at least the crotch region 16. The absorbent core 34 may extend into either or both of the front and rear regions 12, 14. Although the absorbent core 34 depicted in FIG. 1 has a substantially rectangular shape, other shapes may be used, such as a “T” shape or an hourglass shape. The shape of the absorbent core 34 may be selected to provide the greatest absorbency with a reduced amount of material. The absorbent core may be associated with the top sheet 30, back sheet 32, or any other suitable part of the article 10 by any method known in the art, in order to fix the absorbent core 34 in place.

The absorbent core 34 may be made from any suitable material or materials known in the art. Examples of suitable materials for use as the absorbent core 34 include tow fibers, creped cellulose wadding, absorbent foams, absorbent sponges, super absorbent polymers (“SAP”), absorbent gelling materials, fiberized cellulose, fluff pulp having tissue or synthetic materials between the absorbent core 34 and the top sheet 30 or any equivalent material or combination of materials. The size and capacity of the absorbent material should correspond to the application, for example, an incontinent brief for an adult may require a larger absorbent core than a diaper for a child. Zoned absorbency also may be used, if desired. For example, more absorbent capacity may be located in particular regions of the article 10 depending on, for example, the gender of the intended wearer or the intended use for the article 10. The invention is not intended to be limited to any specific materials for use in the absorbent core 34.

In a preferred embodiment, the absorbent core 34 comprises super absorbent polymer distributed within a fibrous structure. Absorbent cores of this type are known in the art, and exemplary absorbent cores are described in U.S. Pat. Nos. 5,281,207 and 6,068,620, issued to Chmielewski et al., and U.S. Pat. No. 5,863,288, issued to Baker, which are herein incorporated by reference in their entirety.

Additional sublayers, transfer layers, acquisition layers, tissue wraps, and the like also may be incorporated into the absorbent core 34, or may be disposed somewhere between top sheet 30 and back sheet 32. Such layers may be provided to assist with transferring fluids to the absorbent core 34, handling fluid surges, preventing rewet, containing absorbent material, improving core stability, or for other purposes. For example, a substantially rectangular, preferably nonwoven, sublayer (not shown), having a basis weight of about 10-200 gm/m², preferably about 40-100 gm/m², may overlay absorbent core 34. Those of ordinary skill in the art are capable of selecting materials, dimensions, and locations for such layers without undue experimentation.

The absorbent core 34 may be any absorbent means that is capable of absorbing or retaining liquids (e.g., menses and/or urine). The absorbent core 34 may be manufactured in a wide variety of sizes and shapes (e.g., rectangular, oval, hourglass, dog bone, asymmetric, etc.).

In a preferred embodiment, the absorbent core 34 is a laminate comprised of a fibrous layer containing superabsorbent polymer material in the form of particles or fibers disposed within a matrix of other hydrophilic fibers, such as cellulose, which may be further be encased in or between first and second tissue layers (or “upper” and “lower” tissue layers). The first and second tissue layers preferably do not contain the superabsorbent polymer material.

The absorbent core 34 may, however, be made from a wide variety of liquid-absorbent materials commonly used in sanitary napkins and other absorbent articles such as comminuted wood pulp that generally is referred to as airfelt or airlaid. Examples of other suitable absorbent materials include creped cellulose wadding; meltblown polymers including COFORM pulp/polypropylene meltblown material (available from Kimberly-Clark Corp., Neenah, Wis.); thermally bonded air-laid fibers; chemically stiffened, modified or cross-linked cellulosic fibers; synthetic fibers such as crimped polyester fibers; peat moss; tissue including tissue wraps and tissue laminates; absorbent foams; absorbent sponges; superabsorbent polymers; absorbent gelling materials; or any equivalent material or combinations of materials, or mixtures of these.

The configuration and construction of the absorbent core 34 also may be varied (e.g., the absorbent core may have varying caliper zones (e.g., profiled so as to be thicker in the center), hydrophilic and/or hydrophobic gradients, superabsorbent gradients, or lower density and higher or lower average basis weight acquisition zones; or may comprise one or more layers or structures). The total absorbent capacity of the absorbent core 34 should, however, be compatible with the design loading and the intended use of the absorbent article. Further, the size and absorbent capacity of the absorbent core 34 may be varied to accommodate different uses such as incontinence pads, pantiliners, regular sanitary napkins, overnight sanitary napkins, regular diapers, overnight diapers, adult diapers, pull-on pants, etc.

Exemplary absorbent structures for use as the absorbent core of the invention are described in U.S. Pat. Nos. 4,950,264, 4,610,678, 4,834,735, 6,068,620, 6,099,950, 6,121,509, 6,171,291, 6,224,961, and European Patent Application No. 0 198 683, the disclosures of which are incorporated by reference herein in their entirety. For example, a thin absorbent core may comprise a low bulk compressed fibrous web layer having super absorbent particles distributed therein.

Referring back to FIG. 1, the crotch region 16 may also include mechanical sealing devices to provide the article 10 with a leak-proof fit around the wearer. In a preferred embodiment, the crotch edges 20 each have one or more elastic gathers 36 may be disposed at or near the crotch edges 20 to help them contract about the contours of the wearer's body, thereby providing a leak-proof seal. Such gathers 36 are known in the art, and are disclosed, for example, in U.S. Pat. No. 5,830,203, issued to Suzuki et al., which is herein incorporated by reference in its entirety in a manner consistent with the invention. The gathers 36 may extend into the rear waist band 14, and may extend as far as the front longitudinally distal edge 18.

In another preferred embodiment, standing leg gathers 38 are disposed on the top sheet 30. Standing leg gathers 38 are strips of material that rise vertically from the surface of the top sheet 30 to provide additional sealing to the article 10. The standing leg gathers 38 may extend across all or part of the article 10 along its longitudinal axis 100. Typically, one standing leg gather 38 is located on either side of the absorbent core 34. The standing leg gathers 38 may be made from folded portions of the top sheet 30 or back sheet 32, or may be made from additional strips of material. Each standing leg gather 38 may be equipped with one or more elastic elements to help seal the gather to the wearer's body. Standing leg gathers are known in the art, and disclosed in U.S. Pat. No. 5,292,316, issued to Suzuki, which is herein incorporated by reference in its entirety in a manner consistent with the invention.

Any suitable elastic material may be used for the gathers 36 and the standing leg gathers 38. Preferably, the elastic material may be stretched to between more than 5% to 500%, and preferably about 200% to about 400%, of its original length without losing its resilience. The elastic material used for the gathers 36 and standing leg gathers 38 may comprise an elastic film, a multidirectional elastic aggregate such as elastic webbing, netting, or scrim elastic, such as FLEXCEL Elastic Nonwoven Laminate, available from Kimberly-Clark Corporation, headquartered in Neenah, Wis., or strands or bands of suitable elastic materials, such as natural or synthetic rubber, urethane elastomers, spandex (e.g., LYCRA, available from E. I. Du Pont de Nemours and Company Corporation, Wilmington, Del.) and elastic polymers. The elastics materials may be attached to the article 10 in any of several ways known in the art. For example, the elastic materials may be ultrasonically bonded, heat/pressure sealed using a variety of bonding patterns, or glued to the diaper 10 using a variety of adhesives. Other performance enhancing devices, such as pockets, baffles, and openings in the top sheet also may be used.

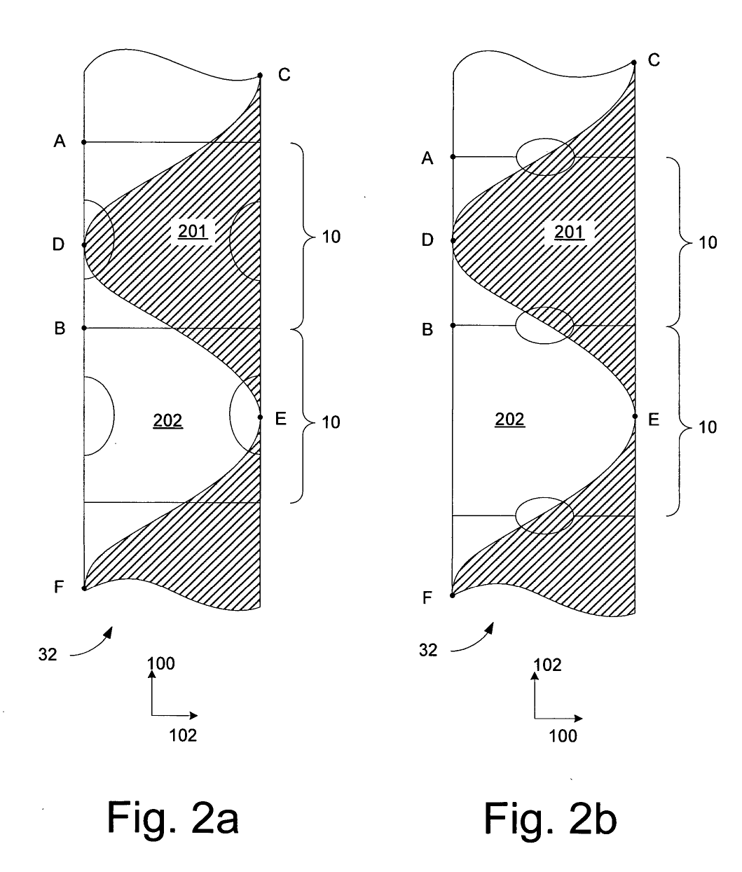

FIGS. 2a and 2b depict views of a decorative component material used to prepare back sheet material 32 with two color schemes disposed on it according to one preferred embodiment, with an overlay outline of cut-outs of absorbent articles 10 that may be formed from the back sheet material 32. The depicted absorbent articles may be cut-out from the back sheet material 32 with either the longitudinal dimension of the absorbent articles 10 substantially corresponding to the longitudinal dimension of the decorative component material (FIG. 2a), or with the lateral dimension of the absorbent articles 10 substantially corresponding to the longitudinal dimension of the decorative component material (FIG. 2b).

In the embodiment seen in FIG. 2a, the longitudinal length of the component prepared from the decorative component material, e.g., the back sheet, as measured in its stretched position is the distance between Points A and B, and the pattern length of the design is the distance between Points C and E for the design designated as 201, and the distance between Points D and F for the design designated as 202. As can be seen, the pattern lengths are greater than the longitudinal length of the back sheets 32, which comport with the longitudinal length of the absorbent articles 10. In the embodiment shown, the decorative component material is provided to the assembly line such that the longitudinal dimension of the material substantially corresponds with the longitudinal dimension of the absorbent articles 10. FIG. 2a demonstrates how successive absorbent articles 10 continuously manufactured using a back sheet 32 made from the decorative component material of the invention will have different decorative appearances.

Alternatively, as seen in FIG. 2b, the lateral length of the component prepared from the decorative component material, e.g., the back sheet, as measured in its stretched position is the distance between Points A and B, and the pattern length of the design is the distance between Points C and E for the design designated as 201, and the distance between Points D and F for the design designated as 202. As can be seen, the pattern lengths are greater than the lateral length of the back sheets 32, which correspond to the lateral length of the absorbent articles 10. In the embodiment shown, the decorative component material is provided to the assembly line such that the longitudinal dimension of the material substantially corresponds with the lateral dimension of the absorbent articles 10. FIG. 2b demonstrates how successive absorbent articles 10 continuously manufactured using a back sheet 32 made from the decorative component material of the invention will have different decorative appearances.

In other embodiments, the pattern length may be considered as the distance between Points C and F, where the design itself comprises multiple designs 201 and 202.

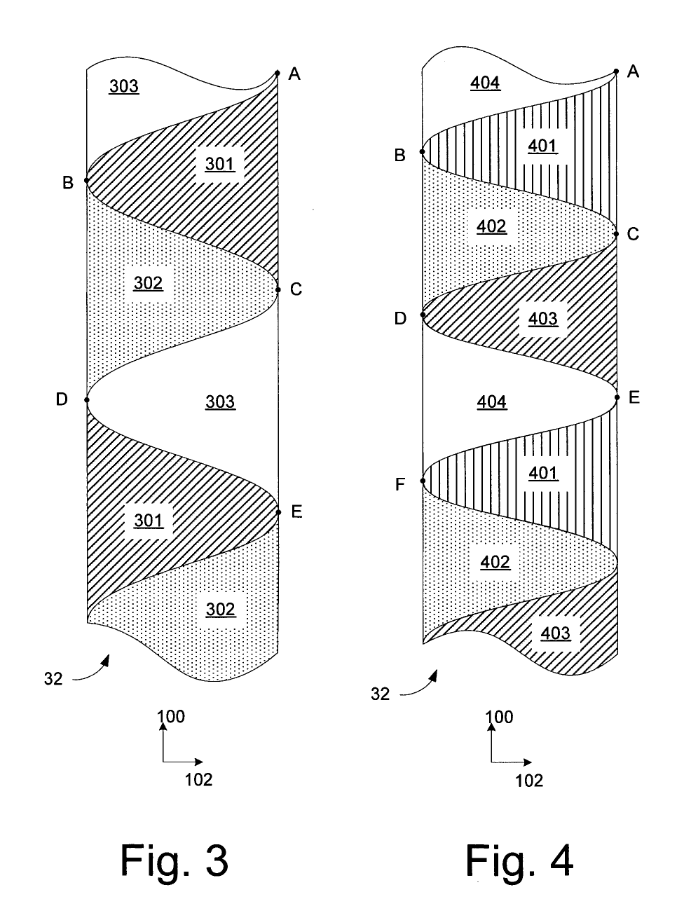

FIGS. 3 and 4 depict exemplary designs that could be used to prepare a back sheet material 32 from a decorative component material according to another preferred embodiment. FIG. 3 comprises three designs, designated as areas 301, 302, and 303, with the distances between Points A and C, Points B and D, and Points C and E, respectively, defining the pattern length of each design. The three designs comprise colors that should be selected such that they complement each other, and further that they blend into each other toward their peripheries. A gradual grading of the tones of the colors along the pattern length could be used to blend the peripheries of adjacent designs.

The design can be considered as comprising multiple designs, which in FIG. 3 is the three (3) designs between Points A and E designated as areas 301, 302, and 303, or alternatively the design may comprise all three areas 301, 302, and 303. The pattern length should be set at a length that does not correspond to one or both of the longitudinal and lateral lengths of the back sheet 32 needed to prepare an absorbent article.

FIG. 4 depicts a design comprising four (4) colors designated as areas 401, 402, 403, and 404, with the distance between Points A and C, Points B and D, Points C and E, and Points D and F, respectively, defining the pattern length of each design. Alternatively, the pattern length could be considered the design defined between Points A and F, where the design comprises all four areas 401, 402, 403, and 404.

In accordance with the preferred embodiments, the pattern length of the design is selected so that it does not correspond to one or both of the longitudinal and lateral lengths of the back sheet material 32 needed to prepare an absorbent article.

The invention is further illustrated by the following Examples which should not be regarded as limiting.

### EXAMPLE 1

A design is disposed on a decorative component material having a longitudinal dimension and opposing lateral edges, the design comprising a pattern including various shades of the three colors of blue, green, and purple with the design as depicted in areas 301, 302, and 303, respectively, of FIG. 3. The design is similar to a sinusoidal curve oriented such that the curve extends along the longitudinal dimension of the decorative component material. The pattern length may be measured as either a full or a half wavelength in the longitudinal dimension of the decorative component material. The base of each curve is oriented substantially coterminous along a lateral edge of the decorative component material, with the apex of each curve fully extending to the opposing lateral edge of the decorative component material. Successive sinusoidal curve patterns as described are alternately disposed on the opposing lateral edges of the decorative component, as seen in FIG. 3, such that the patterns with bases on opposing lateral edges complement each other to completely cover the decorative component material.

The colors of the design are graded in intensity and in hue along the longitudinal dimension of the decorative component material such that the colors blend into the color of the adjacent design at their bordering edges so that the designs give the impression of being one continuous, multicolored pattern. For example, the blue color depicted in area 301 of FIG. 3 varies in shade toward a green hue as it nears the edge of the adjacent green curve depicted as area 302. Similarly, the green curve of area 302 varies in shade toward a purple hue as it nears the edge of the adjacent purple curve of area 303, and the purple curve 303 varies in shade toward a blue hue as it nears the edge of the adjacent blue curve 301.

The longitudinal length of decorative component material needed to prepare a back sheet for an absorbent article may range from about 31 cm for a small sized article to about 54 cm for an extra-large sized article. Thus, to prepare any sized absorbent article using the decorative component material for the back sheet, the design should preferably, but not necessarily, be less than 31 cm, or greater than 54 cm. For example, a design that is 25 cm in longitudinal length, or that is 75 cm in longitudinal length, would be acceptable because these dimensions are either less than or greater than any back sheet longitudinal dimensions, and also are not a multiple of any back sheet longitudinal dimensions.

### EXAMPLE 2

Similar to Example 1, a decorative component material is prepared with a four color design as shown in FIG. 4, the four colors being blue in area 401, purple in area 402, green in area 403, and teal in area 404. Complementary curves are positioned opposite each other along the lateral edges of the decorative component material. Sinusoidal curves are suitable and preferred patterns, but other patterns such as triangles, squares, rectangles and the like may be used.

### EXAMPLE 3

A back sheet material is prepared with a design, such as a tessalation, which is defined as any repeating pattern of interlocking shapes. Several well-known tessalations may be suitable for use with the invention, in particular those tessalations where the designs vary across the pattern length. Using the guidelines provided herein, one of ordinary skill in the art of drawing tessalations can design an appropriate pattern for use in the invention without undue experimentation. The designs may include colors as well.

The same principles apply when a tessalation design is used as when a color design is used. The pattern length of the tessalation must be greater than or less than, but must not correspond to, one or both of the longitudinal and lateral lengths of components made using the decorative component material.

Other embodiments, uses, and advantages of the invention will be apparent to those skilled in the art from consideration of the specification and practice of the invention disclosed herein. The specification should be considered exemplary only, and the scope of the invention is accordingly intended to be limited only by the following claims.

What is claimed is:

1. A decorative component material having a longitudinal dimension for use in the manufacture of an absorbent article, the decorative component material comprising a surface; wherein the surface comprises at least one design, the design having a pattern length in the longitudinal dimension of the decorative component material that is greater than or less than the total length of at least one dimension of at least one decorative component, wherein the decorative component is associated with the decorative component material.

2. The decorative component material of claim 1, wherein the at least one dimension of the decorative component comprises the longitudinal dimension of the decorative component.

3. The decorative component material of claim 1, wherein the at least one dimension of the decorative component comprises the lateral dimension of the decorative component.

4. The decorative component material of claim 1, wherein the decorative component material comprises a back sheet for a disposable absorbent article.

5. The decorative component material of claim 4, wherein the back sheet comprises a material selected from the group consisting of polyethylene, polypropylene, polyester, nylon, polylactic acid, polyvinyl chloride and blends of these materials.

6. The decorative component material of claim 5, wherein the back sheet has disposed thereon an additional layer comprising a material selected from the group consisting of spun-bonded nonwoven web of synthetic fibers; a nonwoven web of cellulosic fibers, textile fibers, or a blend of cellulosic and textile fibers; a spun-bonded nonwoven web of synthetic fibers mixed with cellulosic, pulp fibers, or textile fibers; and melt blown thermoplastic fibers or mixtures of such thermoplastic fibers with cellulosic, pulp or textile fibers.

7. The decorative component material of claim 1, wherein the design is printed on the decorative component material using an offset printing process.

8. A method of making an absorbent article, the absorbent article having a longitudinal dimension substantially corresponding to the front-to-rear dimension of the wearer of the absorbent article, comprising:
providing a back sheet material;
providing at least one decorative component on the back sheet material, the decorative component comprising at least one design disposed in at least one dimension of the decorative component, the design having a pattern length that is not substantially equal to the total length of the at least one dimension of the decorative component;
providing a top sheet material; and
providing an absorbent core at least partially between the back sheet material and the top sheet material.

9. The method of claim 8, wherein the at least one dimension of the decorative component comprises the longitudinal dimension of the decorative component.

10. The method of claim 8, wherein the at least one dimension of the decorative component comprises the lateral dimension of the decorative component.

11. The method of claim 8, wherein the the back sheet material is liquid impervious.

12. The method of claim 8, wherein the backsheet is liquid pervious.

13. The method of claim 8 wherein the back sheet comprises a material selected from the group consisting of polyethylene, polypropylene, polyester, nylon, and polyvinyl chloride, polylactic acid and blends of these materials.

14. The method of claim 13, wherein the back sheet has disposed thereon an additional layer comprising a material selected from the group consisting of spun-bonded nonwoven web of synthetic fibers; a nonwoven web of cellulosic fibers, textile fibers, or a blend of cellulosic and textile fibers; a spun-bonded nonwoven web of synthetic fibers mixed with cellulosic, pulp fibers, or textile fibers; and melt blown thermoplastic fibers or mixtures of such thermoplastic fibers with cellulosic, pulp or textile fibers.

15. The method of claim 8, wherein the pattern is printed on the material comprising the decorative component using an offset printing process.

16. A method of preparing a package of absorbent articles with varied decorative appearances, comprising:
providing a back sheet material to an assembly line;
providing a top sheet material;
providing an absorbent core at least partially between the back sheet material and the top sheet material; and
assembling a plurality of absorbent articles into a package of absorbent articles,
wherein the absorbent article further comprises at least one decorative component, the decorative component comprising at least one design disposed in at least one dimension of the decorative component, the design having a pattern length that is not substantially equal to the total length of the at least one dimension of the decorative component.

17. The method of claim 16, wherein the at least one dimension of the decorative component comprises the longitudinal dimension.

18. The method of claim 16, wherein the at least one dimension of the decorative component comprises the lateral dimension.

19. The method of claim 16, wherein the decorative component comprises the back sheet.

20. The method of claim 19, wherein the back sheet comprises a material selected from the group consisting of polyethylene, polypropylene, polyester, nylon, and polyvinyl chloride, polylactic acid and blends of these materials.

21. The method of claim 20, wherein the back sheet has disposed thereon an additional layer comprising a material selected from the group consisting of spun-bonded nonwoven web of synthetic fibers; a nonwoven web of cellulosic fibers, textile fibers, or a blend of cellulosic and textile fibers; a spun-bonded nonwoven web of synthetic fibers mixed with cellulosic, pulp fibers, or textile fibers; and melt blown thermoplastic fibers or mixtures of such thermoplastic fibers with cellulosic, pulp or textile fibers.

22. The method of claim 16, wherein the design is printed on the decorative component using the offset printing process.
